(19) United States
(12) Patent Application Publication
(10) Pub. No.: US 2007/0142401 A1
(43) Pub. Date: Jun. 21, 2007

(54) Indolyl-pyrroledione derivatives for the treatment of neurological and vascular disorders related to beta-amyloid generation and/or aggregation

(75) Inventors: Yves Auberson, Allschwil (CH); Graeme Bilbe, Neuchatel (CH); Rainer R. Kuhn, Riehen (CH); Peter Von Matt, Biel-Benken (CH); Heinrich Rueger, Flueh (CH); Matthias Staufenbiel, Lorrach (DE); Jurgen Wagner, Bottmingen (CH); Kaspar Zimmermann, Oberwil BL (CH)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 10/576,176

(22) PCT Filed: Oct. 26, 2004
(86) PCT No.: PCT/EP04/12082

§ 371 Date: Nov. 1, 2006

(30) Foreign Application Priority Data
Oct. 27, 2003 (GB) 0325032.1
Oct. 28, 2003 (GB) 0325176.6

(51) Int. Cl. A61K 31/519; A61K 31/506; A61K 31/4709; A61K 31/404

(52) U.S. Cl. 514/256; 514/266.2; 514/314; 514/414

(57) ABSTRACT

The invention relates to the use of an inhibitor of formula (I), or a pharmaceutically acceptable salt thereof having an activity on protein kinases PKC alpha, PKC beta, PKC gamma, PKC epsilon, PKC theta, CDK-1, KDR, PKA, Flt-1, Flt-2, Flt-3 or Flt-4, or on a combination of the above enzymes, for the treatment and/or prevention of neurological and vascular disorders related to beta-amyloid generation and/or aggregation such as neurodegenerative diseases like Down's Syndrome, memory and cognitive impairment, dementia, amyloid neuropathies, brain inflammation, nerve and brain trauma, vascular amyloidosis, or cerebral hemorrhage with amyloidosis.

The invention relates to the use of compounds (hereinafter: “COMPOUND”) or a N-Oxide or a pharmaceutically acceptable salt thereof having an activity on protein kinases PKC alpha, PKC beta, PKC gamma, PKC epsilon, PKC theta, CDK-1, KDR, PKA, Flt-1, Flt-2, Flt-3 or Flt-4, or on a combination of the above enzymes, for the treatment and/or prevention of neurological and vascular disorders related to beta-amyloid generation and/or aggregation such as neurodegenerative diseases like Down's Syndrome, memory and cognitive impairment, dementia, amyloid neuropathies, brain inflammation, nerve and brain trauma, vascular amyloidosis, or cerebral hemorrhage with amyloidosis.

A compound of formula I

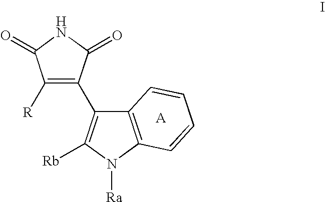

wherein

Rₐ is H; C₁₋₄alkyl; or C₁₋₄alkyl substituted by OH, NH₂, NHC₁₋₄alkyl or N(di-C₁₋₄alkyl)₂;

R_{b} is H; or C₁₋₄alkyl;

R is a radical of formula (a), (b), (c), (d), (e) or (f)

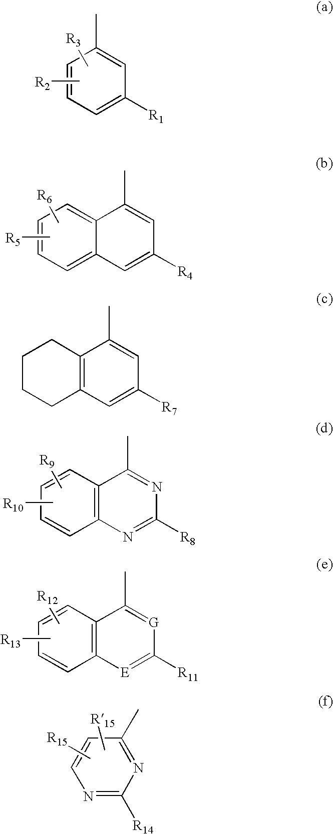

wherein
each of R₁, R₄, R₇, R₈, R₁₁ and R₁₄ is OH; SH; a heterocyclic residue; NR₁₆R₁₇ wherein each of R₁₆ and R₁₇, independently, is H or C₁₋₄alkyl or R₁₆ and R₁₇ form together with the nitrogen atom to which they are bound a heterocyclic residue; or a radical of formula α
   —X—R_{c}—Y (α)
wherein X is a direct bond, O, S or NR₁₈ wherein R₁₈ is H or C₁₋₄alkyl,
R_{c} is C₁₋₄alkylene or C₁₋₄alkylene wherein one CH₂ is replaced by CRₓR_{y} wherein one of Rₓ and R_{y} is H and the other is CH₃, each of Rₓ and R_{y} is CH₃ or Rₓ and R_{y} form together —CH₂—CH₂—, and
Y is bound to the terminal carbon atom and is selected from OH, a heterocyclic residue and —NR₁₉R₂₀ wherein each of R₁₉ and R₂₀ independently is H, C₃₋₆cycloalkyl, C₃₋₆cycloalkyl-C₁₋₄alkyl, aryl-C₁₋₄alkyl or C₁₋₄alkyl optionally substituted on the terminal carbon atom by OH, or R₁₀ and R₂₀ form together with the nitrogen atom to which they are bound a heterocyclic residue;

each of R₂, R₃, R₅, R₆, R₉, R₁₀, R₁₂, R₁₃, R₁₅ and R′₁₅, independently, is H, halogen, C₁₋₄alkyl, CF₃, OH, SH, NH₂, C₁₋₄alkoxy, C₁₋₄alkylthio, NHC₁₋₄alkyl, N(di-C₁₋₄alkyl)₂ or CN;

either E is —N═ and G is —CH═ or E is —CH═ and G is —N═; and

ring A is optionally substituted,

or a salt thereof.

Preferably a compound of formula I wherein the heterocyclic residue as R₁, R₄, R₇, R₈, R₁₁, R₁₄ or Y or formed, respectively, by NR₁₆R₁₇ or NR₁₉R₂₀, is a three to eight membered saturated, unsaturated or aromatic heterocyclic ring comprising 1 or 2 heteroatoms, and optionally substituted on one or more ring carbon atoms and/or on a ring nitrogen atom when present.

More preferably a compound of formula I wherein the heterocyclic residue is R₁, R₄, R₇, R₈, R₁₁, R₁₄ or Y or formed, respectively, by NR₁₆R₁₇ or NR₁₉R₂₀, is a residue of formula (γ).

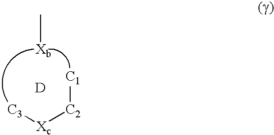

wherein

the ring D is a 5, 6 or 7 membered saturated, unsaturated or aromatic ring;

X_{b} is —N—, —C— or —CH—;

X_{c} is —N═, —NR_{f}—, —CR_{f}′═ or —CHR_{f}′— wherein R_{f} is a substituent for a ring nitrogen atom and is selected from C₁₋₆alkyl; acyl; C₃₋₆cycloalkyl; C₃₋₆cycloalkyl-C₁₋₄alkyl; phenyl; phenyl-C₁₋₄alkyl;

a heterocyclic residue; and a residue of formula β
—R₂₁—Y′ (β)

wherein R₂₁ is C₁₋₄alkylene or C₂₋₄alkylene interrupted by O and Y′ is OH, NH₂, NH(C₁₋₄alkyl) or N(C₁₋₄alkyl)₂; and R_{f}′ is a substituent for a ring carbon atom and is selected from C₁₋₄alkyl;

C₃-cycloalkyl optionally further substituted by C₁₋₄-alkyl; 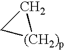
wherein p is 1, 2 or 3; CF_{3;}

halogen; OH; NH₂; —CH₂—NH₂; —CH₂—OH; piperidin-1-yl; and pyrrolidinyl;

the bond between C₁ and C₂ is either saturated or unsaturated;

each of C₁ and C₂, independently, is a carbon atom which is optionally substituted by one or two substituents selected among those indicated above for a ring carbon atom; and

the line between C₃ and X_{b} and between C₁ and X_{b}, respectively, represents the number of carbon atoms as required to obtain a 5, 6 or 7 membered ring D.

Even more preferably a compound of formula I, wherein D is a piperazinyl ring optionally C— and/or N-substituted as specified in claim 3.

Yet even more preferably COMPOUND a compound of formula I wherein

Ra is H; CH₃; CH₂—CH₃; or isopropyl,

Rb is H; halogen; C₁₋₆-alkoxy; or C₁₋₆alkyl, and either

I. R is a radical of formula (a) 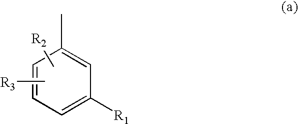
wherein
R₁ is piperazin-1-yl optionally substituted by CH₃ in position 3 or 4; or 4,7-diaza-spiro (2.5] oct-7-yl;
R₂ is Cl; Br; CF₃; or CH₃; and
R₃ is H; CH₃; or CF₃; R₃ being other than H when Ra is H or CH₃, Rb is H and R₁ is 4-methyl-1-piperazinyl; or

II. R is a radical of formula (b) 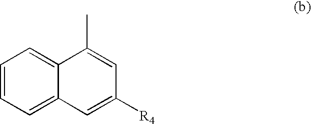
wherein
R₄ is piperazin-1-yl substituted in positions 3 and/or 4 by CH₃; or 4,7-diaza-spiro [2.5] oct-7-yl; Ra being other than H or CH₃ when R₄ is 4-methyl-1-piperazinyl; or

III. R is a residue of formula (c) 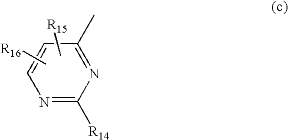
wherein
R₁₄ is piperazin-1-yl optionally substituted by CH₃ in position 3 and/or 4 or in position 3 by ethyl, phenyl-C₁₋₄alkyl, C₁₋₄alkoxy-C₁₋₄alkyl or halogeno-C₁₋₄alkyl; or 4,7-diaza-spiro [2.5] oct-7-yl;
R₁₅ is halogen; CF₃; or CH₃; R₁₅ being other than CH₃ when Ra is H or CH₃, Rb is H and R₁₄ is 4-methyl-1-piperazinyl; and
R₁₆ is H; CH₃; or CF₃; R₁₆ being other than H when R₁₅ is Cl, Ra is H or CH₃, Rb is H and R₁₄ is 4-methyl-1-piperazinyl; or

IV. R is a radical of formula (d) 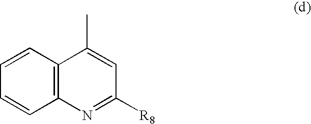
wherein R₈ is piperazin-1-yl, 3-methyl-piperazin-1-yl or 4-benzyl-piperazin-1-yl; or

V. R is a radical of formula (e) 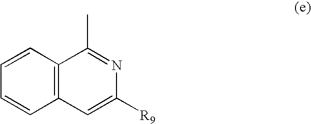
wherein R₉ is 4,7-diaza-spiro [2.5] oct-7-yl; or piperazin-1-yl substituted in position 3 by methyl or ethyl and optionally in position 4 by methyl.

The compounds of formula I may exist in free form or in salt form, e.g. addition salts with e.g. organic or inorganic acids, for example, hydrochloric acid, acetic acid, trifluoroacetic acid. It will be appreciated that the compounds of formula I may exist in the form of optical isomers, racemates or diastereoisomers. For example, a ring carbon atom bearing a substituent in the position 3 of the piperazinyl residue is asymmetric and may have the D- or

L- configuration. It is to be understood that the present invention embraces all enantiomers and their mixtures. Similar considerations apply in relation to starting materials exhibiting asymmetric carbon atoms as mentioned.

An especially preferred COMPOUND is a compound of formula I, as herein before described,

wherein

when R is of formula (a)

R₁ is -(4-methyl-piperazin-1-yl), 1-piperazinyl, 3-methyl-piperazin-1-yl or -(4,7-diaza-spiro[2.5]oct-7-yl)

R₂ is 2-Cl or 2-CH₃

R₃ is 3-CH₃, 3-CF₃or H

Rₐ is H or CH₃

And when,

R is of formula (b)

R₄ is -(4,7-diaza-spiro[2.5]oct-7-yl), 3-methyl-piperazin-1-yl or 4-methyl-3-methyl-piperazin-1-yl

Rₐ is H or CH₃

And when

R is of formula (c)

R₁₄ is -4-methyl-piperazin-1-yl, 3-methyl-piperazin-1-yl, -4,7-diaza-spiro[2.5]oct-7-yl, 1-piperazinyl, 4-methyl-3-methyl-piperazin-yl, 3-methoxyethyl-piperazin-1-yl, 3-ethyl-piperazin-1-yl, 3-benzyl-piperazin-1-yl or 3-CH₂F-piperazin-1-yl

R₁₅ is Cl, Br, CF₃, F

R₁₆ is CH₃, H, CH₂—CH₃

Rₐ is H or CH₃

R_{b} is H, CH₂—CH₂—CH₃, F, CH(CH₃)₂, Cl, OCH₃, CH₃ or CH₂—CH₃

And when

R is of formula (d)

R₈ is 3-methyl-piperazin-1-yl, 4-benzyl-1-piperazinyl or 1-piperazinyl

Rₐ is CH₃ or H

And when

R is of formula (e)

R₉ is -4,7-diaza-spiro[2.5]oct-7-yl, 3-ethyl-piperazin-1-yl, 3-methyl-piperazin-1-yl, 4-methyl-3-methyl-piperazin-1-yl or 3-ethyl-piperazin-1-yl

Rₐ is H, CH₂—CH₃ or CH(CH₃)₂

R_{b} is CH₃, F, CH(CH₃)₂, OCH₃, CH₂—CH₃ or Cl

most prefered COMPOUND is 3-[2-Chloro-5-(4-methyl-piperazin-1-yl)-3-trifluoromethyl-phenyl]-4-(1H-indol-3-yl)-pyrrole-2,5-dione having the formula

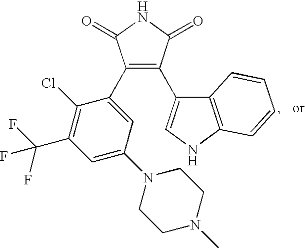

3-(1H-Indol-3-yl)-4-[2-(4-methyl-piperazin-1-yl)-quinazolin-4-yl]-pyrrole-2,5-dione having the formula

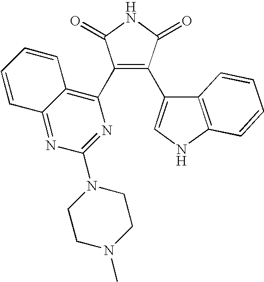

Even more preferred, Compound means any of the other definitions of COMPOUND wherein the compound has an activity on PKC alpha, PKC beta, PKC gamma, PKC epsilon, PKC theta, or on a combination of these enzymes.

Compounds of formula I and methods for the preparation of such compounds are in particular generically and specifically disclosed in the patents and patent application WO2003082859, in particular in the compound claims and the final products of the working examples, the subject-matter of the final products, the pharmaceutical preparations and the claims is hereby incorporated into the present application by reference to this publication.

The general terms used hereinbefore and hereinafter preferably have within the context of this disclosure the following meanings, unless otherwise indicated:

Alkyl or alkoxy may be straight or branched. Phenyl-C₁₋₄alkyl is preferably benzyl or phenethyl. In C₁₋₄alkoxy-C₁₋₄alkyl the alkoxy moiety is preferably methoxy or ethoxy and the alkyl moiety preferably methyl or ethyl; a suitable example is e.g. 2-methoxyethyl. Halogen may be F, Cl, Br or I, preferably F, Cl or Br. Halogeno-C₁₋₄alkyl is alkyl wherein one or more H are replaced by halogen, e.g. Cl or F, e.g. CH₂Cl, CH₂F or CF₃
R is preferably a radical of formula (a), (c) or (e).

In the radical of formula (a) or (c), R₂ or R₁₅ is preferably in para to R₁ or R₁₄, respectively. R₃ is preferably in meta to R₁. In the radical or formula (e), R₉ is preferably 4,7-diaza-spiro [2.5] oct-7-yl.

PKC is protein kinase C

CDK is cyclin dependent kinase

PKA is protein kinase A

Salts are especially the pharmaceutically acceptable salts of compounds of formula I.

Such salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

The invention further relates to the use of COMPOUND or a N-Oxide or a pharmaceutically acceptable salt thereof for the manufacture of medicament having an activity on protein kinases PKC alpha, PKC beta, PKC gamma, PKC epsilon, PKC theta, CDK-1, KDR, PKA, Flt-1, Flt-2, Flt-3 or Flt-4, or on a combination of the above enzymes, for the treatment and/or prevention of neurological and vascular disorders related to beta-amyloid generation and/or aggregation such as neurodegenerative diseases like Down's Syndrome, memory and cognitive impairment, dementia, amyloid neuropathies, brain inflammation, nerve and brain trauma, vascular amyloidosis, or cerebral hemorrhage with amyloidosis.

Depending on species, age, individual condition, mode of administration, and the clinical picture in question, effective doses, for example daily doses of about 10-1000 mg, preferably 10-50 mg or 50-200 mg or 200-400 mg, especially 50-100 mg or 300-400 mg, are administered to warm-blooded animals of about 70 kg bodyweight. For adult patients with neurological and vascular disorders related to beta-amyloid generation and/or aggregation, especially neurodegenerative diseases like Down's Syndrome, memory and cognitive impairment, dementia, amyloid neuropathies, brain inflammation, nerve and brain trauma, vascular amyloidosis, or cerebral hemorrhage with amyloidosis.

The invention relates likewise to a process or a method for the treatment of neurological and vascular disorders related to beta-amyloid generation and/or aggregation, especially neurodegenerative diseases like Down's Syndrome, memory and cognitive impairment, dementia, amyloid neuropathies, brain inflammation, nerve and brain trauma, vascular amyloidosis, or cerebral hemorrhage with amyloidosis. The COMPOUNDS thereof can be administered as such or especially in the form of pharmaceutical compositions, prophylactically or therapeutically, preferably in an amount effective against the said diseases, to a warm-blooded animal, for example a human, requiring such treatment. In the case of an individual having a bodyweight of about 70 kg the daily dose administered is from approximately 0.01 g to approximately 5 g, preferably from approximately 0.25 g to approximately 1.5 g, more preferably 0.01 g to 0.05 g, even more preferably 0.025 g to 0.1 g most preferably 0.05 g to 1 g of a compound of the present invention.

The compounds of formula I may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. Pharmaceutical compositions comprising a compound of formula I in free form or in pharmaceutically acceptable salt form in association with at least one pharmaceutical acceptable carrier or diluent may be manufactured in conventional manner by mixing with a pharmaceutically acceptable carrier or diluent. Unit dosage forms for oral administration contain, for example, from about 0.1 mg to about 500 mg of active substance.

Topical administration is e.g. to the skin. A further form of topical administration is to the eye. The compounds of formula I may be administered in free form or in pharmaceutically acceptable salt form e.g. as indicated above. Such salts may be prepared in conventional manner and exhibit the same order of activity as the free compounds.

The invention relates also to a method for administering to a human subject suffering from a neurological and vascular disorders related to beta-amyloid generation and/or aggregation, especially neurodegenerative diseases like Down's Syndrome, memory and cognitive impairment, dementia, amyloid neuropathies, brain inflammation, nerve and brain trauma, vascular amyloidosis, or cerebral hemorrhage with amyloidosis, COMPOUND or a pharmaceutically acceptable salt thereof, which comprises administering a pharmaceutically effective amount of COMPOUND or a pharmaceutically acceptable salt thereof to the human subject, preferably once daily for a period exceeding 3 months. The invention relates especially to such method wherein a daily dose of 200 to 800 mg, or 10 mg to 200 mg especially 400-600 mg or 10-100 mg, preferably 400 mg or 10-50 mg, of COMPOUND is administered.

The invention also relates in a combination which comprises (a) COMPOUND or a pharmaceutically acceptable salt thereof and (b) a therapeutic agent for the treatment of neurological and vascular disorders related to beta-amyloid generation and/or aggregation, most preferably a combination wherein the combination partners are present in synergistically effective amounts.

The effective dosage of each of the combination partners employed in the combination may vary depending on a variety of factors including the particular combination of the pharmaceutical compound partners, the route of administration, the severity of the disease, the renal and hepatic functions of the patient. The molar ratio (a)/(b) of the combination partners is about 0.1 to 10, most preferably 0.3 to 3 and the unit dosage form contains 20 to 200 mg, most preferably 50 to 150 mg of 3-[2-Chloro-5-(4-methyl-piperazin-1-yl)-3-trifluoromethyl-phenyl]-4-(1H-indol-3-yl)-pyrrole-2,5-dione of the formula I.

### EXAMPLE 1

Cell Culture

HEK/APPswe cells are plated in microtiter plates precoated with 10 μg/ml poly-D-lysine at 12′000 cells/well in 100 μl/well DMEM medium supplemented with 10% FCS, 0.25 mg/ml G418 sulfate, 1% penicillin streptomycin. The following day, supernatant is replaced with 90 μl/well of fresh medium and 10 μl/well of compound diluted in culture medium are added. Two types of control wells are used: cell culture medium without cells plus 10 μl/well of all compound dilutions (background signals) and cell culture medium from untreated cells (positive control). 24 hours later after compound addition, conditioned medium is collected and Aβ levels determined by a specific sandwich ELISA.

Aβ₄₀ and Aβ₄₂ Detection by Sandwich ELISA

For the sandwich ELISA, the maxisorp microtiterplates are coated overnight at 4° C. with 100 μl/well of the monoclonal antibody 25H 10 diluted 1:1000 in PB for Aβ₄₀ detection or monoclonal antibody B10E7 diluted 1:2750 for detection of Aβ₄₂. Wells are then emptied, washed three times with 350 μl PBS and blocking is performed for 2 hours at room temperature with 200 μl/well of 2% BSA, 0.05% Tween20 in PBS. After washing the wells as described above, 10 μl of the conditioned media samples to be tested are added to wells containing 90 μl of medium and 0.18 μg/ml of biotinylated monoclonal β1 antibody and incubated overnight at 4° C. Wells were washed as described above and 100 μl/well of alkaline phosphatase coupled to streptavidin diluted 1:5′000 in medium are added. After 1 hour incubation at room temperature wells are washed as described above and alkaline phosphatase activity is determined by adding 100 μl/well of diethanolamine buffer, pH 9.8 (100 mM diethanolamine, 1 mM MgCl₂, pH adjusted to 9.8 with 2 M HCl) containing the chemiluminescent CSPD substrate (25 mM stock solution diluted 1:416) and the enhancer Emerald II (diluted 1:10). After 15 minutes incubation at room temperature in the dark, plates are measured on the luminometer (Analyst AD; LJL Biosystems, USA Aβ₄₀). Values are given as % reduction of A β̅. The 100% reduction value is calculated from a series of wells containing only medium and extract and the 0% reduction value from conditioned medium only. Samples are measured in triplicate. A reference compound is included in all plates as control for assay performance.

MTS Assay

To determine cytotoxicity, cells are tested by the MTS colorimetric kit performed essentially according to the manufacturer's specifications (Promega, #G5430X). After collecting the conditioned medium for the sandwich ELISA, the rest of the conditioned medium is removed completely and replaced with 100 μl/well culture medium containing one fifth of MTS solution prepared as recommended in the kit. After 3 hours incubation at 37° C., absorbance is read at an OD of 490 nm with a reference wavelength set to 630 nm. Values are given as % metabolic rate (n=6). The 0% value is calculated from wells which had no cells, 100% from wells with an untreated cell layer

### EXAMPLE 2

### 3-[2-Chloro-5-(4-methyl-piperazin-1-yl)-3-trifluoromethyl-phenyl]-4-(1H-indol-3-yl)-pyrrole-2,5-dione

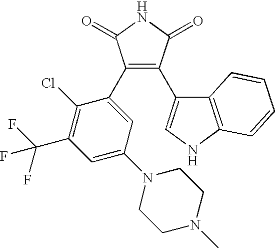

This compound has the following activities in cell-free enzyme assays:

| | | |
|---|---|---|
| PKC alpha | 21 | nM |
| PKC beta | 30 | nM |
| PKC gamma | <500 | nM |
| PKC epsilon | 514 | nM |
| PKC theta | 186 | nM |
| CDK-1 | <10 | microM |
| KDR | <10 | microM |
| PKA | <10 | microM |
| Flt-1 | <10 | microM |
| Flt-2 | <10 | microM |
| Flt-3 | <10 | microM |
| Flt-4 | <10 | microM |

The compound of Example 2 demonstrates a clear reduction of Aβ secretion in the medium of HEK/APPswe cell cultures at concentrations below 1 microM, without having any effect on cellular viability.

1. A method for the treatment and/or prevention of neurological and vascular disorders related to beta-amyloid generation and/or aggregation comprising administering an inhibitor of one or more of protein kinases PKC alpha, PKC beta, PKC gamma, PKC epsilon, PKC theta, CDK-1, KDR, PKA, Flt-1, Flt-2, Flt-3 and Flt-4.

2. The method according to claim 1 wherein the inhibitor is a compound of formula I 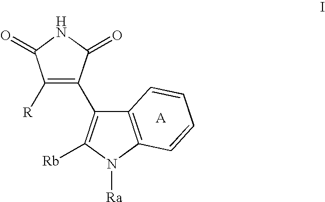
wherein
Rₐ is H; C₁₋₄alkyl; or C₁₋₄alkyl substituted by OH, NH₂, NHC₁₋₄alkyl or N(di-C₁₋₄alkyl)₂;
R_{b} is H; or C₁₋₄alkyl;
R is a radical of formula (a), (b), (c), (d), (e) or (f) 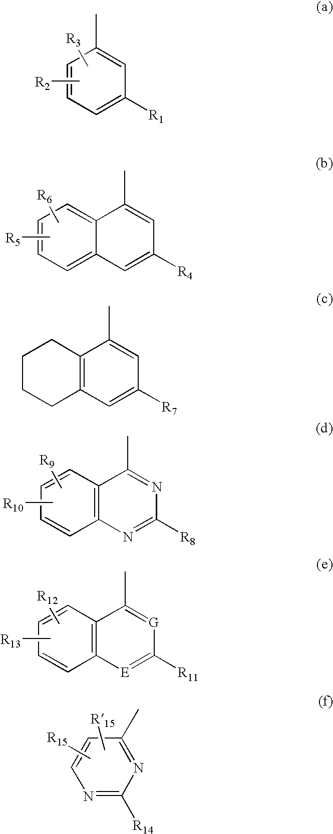
wherein
each of R₁, R₄, R₇, R₈, R₁₁, and R₁₄ is OH; SH; a heterocyclic residue; NR₁₆R₁₇ wherein each of R₁₆ and R₁₇, independently, is H or C₁₋₄alkyl or R₁₆ and R₁₇ form together with the nitrogen atom to which they are bound a heterocyclic residue; or a radical of formula α
—X—R_{c}—Y (α)
wherein X is a direct bond, O, S or NR₁₈ wherein R₁₈ is H or C₁₋₄alkyl,
R_{c} is C₁₋₄alkylene or C₁₋₄alkylene wherein one CH₂ is replaced by CRₓR_{y} wherein one of Rₓ and R_{y} is H and the other is CH₃, each of Rₓ and R_{y} is CH₃ or Rₓ and R_{y} form together —CH₂—CH₂—, and
Y is bound to the terminal carbon atom and is selected from OH, a heterocyclic residue and —NR₁₉R₂₀ wherein each of R₁₉ and R₂₀ independently is H, C₃₋₆cycloalkyl, C₃₋₆cycloalkyl-C₁₋₄alkyl, aryl-C₁₋₄alkyl or C₁₋₄alkyl optionally substituted on the terminal carbon atom by OH, or R₁₉ and R₂₀ form together with the nitrogen atom to which they are bound a heterocyclic residue;
each of R₂, R₃, R₅, R₆, R₉, R₁₀, R₁₂, R₁₃, R₁₅ and R′₁₅, independently, is H, halogen, C₁₋₄alkyl, CF₃, OH, SH, NH₂, C₁₋₄alkoxy, C₁₋₄alkylthio, NHC₁₋₄alkyl, N(di-C₁₋₄alkyl)₂ or CN;
either E is —N═ and G is —CH═ or E is —CH═ and G is —N═;
or a salt thereof.

3. A method according to claim 2, wherein the heterocyclic residue as R₁, R₄, R₇, R₈, R₁₁, R₁₄ or Y or formed, respectively, by NR₁₆R₁₇ or NR₁₉R₂₀, is a three to eight membered saturated, unsaturated or aromatic heterocyclic ring comprising 1 or 2 heteroatoms, and optionally substituted on one or more ring carbon atoms and/or on a ring nitrogen atom when present.

4. A method according to claim 2, wherein the heterocyclic residue as R₁, R₄, R₇, R₈, R₁₁, R₁₄ or Y or formed, respectively, by NR₁₆R₁₇ or NR₁₉R₂₀, is a residue of formula (γ) 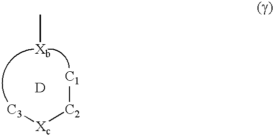
wherein
the ring D is a 5, 6 or 7 membered saturated, unsaturated or aromatic ring;
X_{b} is —N—, —C═ or —CH—;
X_{c} is —N═, —NR_{f}—, —CR_{f}′═ or —CHR_{f}′— wherein R_{f} is a substituent for a ring nitrogen atom and is selected from C₁₋₆alkyl; acyl; C₃₋₆cycloalkyl; C₃₋₆cycloalkyl-C₁₋₄alkyl; phenyl; phenyl-C₁₋₄alkyl heterocyclic residue; and a residue of formula β
—R₂₁—Y′ (β)
wherein R₂₁ is C₁₋₄alkylene or C₂₋₄alkylene interrupted by O and Y′ is OH, NH₂, NH(C₁₋₄alkyl) or N(C₁₋₄alkyl)₂; and R_{f}′ is a substituent for a ring carbon atom and is selected from C₁₋₄alkyl; C₃₋₆cycloalkyl optionally further substituted by C₁₋₄alkyl; 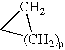
wherein p is 1, 2 or 3; CF₃;
halogen; OH; NH₂; —CH₂—NH₂; —CH₂—OH; piperidin-1-yl; and pyrrolidinyl;
the bond between C₁ and C₂ is either saturated or unsaturated;
each of C₁ and C₂, independently, is a carbon atom which is optionally substituted by one or two substituents selected among those indicated above for a ring carbon atom; and
the line between C₃ and X_{b} and between C₁ and X_{b}, respectively, represents the number of carbon atoms as required to obtain a 5, 6 or 7 membered ring D.

5. A method according to claim 4, wherein D is a piperazinyl ring optionally C— and/or N-substituted as specified in claim 4.

6. A method according to claim 2, wherein
Ra is H; CH₃; CH₂—CH₃; or isopropyl,
Rb is H; halogen; C₁₋₆alkoxy; or C₁₋₆alkyl, and either
I. R is a radical of formula (a) 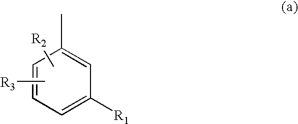
wherein
R₁ is piperazin-1-yl optionally substituted by CH₃ in position 3 or 4; or 4,7-diaza-spiro [2.5] oct-7-yl;
R2 is Cl; Br; CF₃; or CH₃; and
R3 is H; CH₃; or CF₃; R₃ being other than H when Ra is H or CH₃, Rb is H and R₁ is 4-methyl-1-piperazinyl; or
II. R is a radical of formula (b) 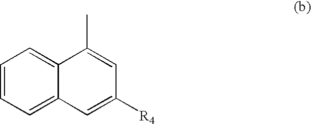
wherein
R₄ is piperazin-1-yl substituted in positions 3 and/or 4 by CH₃; or 4,7-diaza-spiro [2.5] oct-7-yl; Ra being other than H or CH₃ when R₄ is 4-methyl-1-piperazinyl; or
III. R is a residue of formula (c) 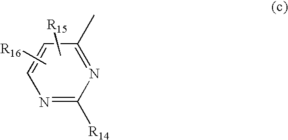
wherein
R₁₄ is piperazin-1-yl optionally substituted by CH₃ in position 3 and/or 4 or in position 3 by ethyl, phenyl-C₁₋₄alkyl, C₁₋₄alkoxy-C₁₋₄alkyl or halogeno-C₁₋₄alkyl; or 4,7-diaza-spiro [2.5] oct-7-yl;
R₁₅ is halogen; CF₃; or CH₃; R₁₅ being other than CH₃ when Ra is H or CH₃, Rb is H and
R₁₄ is 4-methyl-1-piperazinyl; and
R₁₆ is H; CH₃; or CF₃; R₁₆ being other than H when R₁₅ is Cl, Ra is H or CH₃, Rb is H and R₁₄ is 4-methyl-1-piperazinyl; or
IV. R is a radical of formula (d) 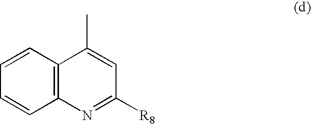
wherein R₈ is piperazin-1-yl, 3-methyl-piperazin-1-yl or 4-benzyl-piperazin-1-yl; or
V. R is a radical of formula (e) 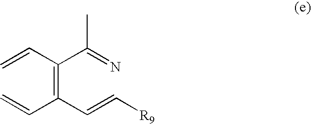
wherein R₉ is 4,7-diaza-spiro [2.5] oct-7-yl; or piperazin-1-yl substituted in position 3 by methyl or ethyl and optionally in position 4 by methyl; or a pharmaceutically acceptable salt thereof.

7. A method according to claim 1, wherein
when R is of formula (a)
R₁ is -(4-methyl-piperazin-1-yl), 1-piperazinyl, 3-methyl-piperazin-1-yl or -(4,7-diaza spiro[2.5]oct-7-yl)
R₂ is 2-Cl or 2-CH₃
R₃ is 3-CH₃, 3-CF₃or H
Rₐ is H or CH₃
And when,
R is of formula (b)
R₄ is -(4,7-diaza-spiro[2.5]oct-7-yl), 3-methyl-piperazin-1-yl or 4-methyl-3-methyl-piperazin-1-yl
Rₐ is H or CH₃
And when
R is of formula (c)
R₁₄ is -4-methyl-piperazin-1-yl, 3-methyl-piperazin-1-yl, -4,7-diaza-spiro[2.5]oct-7-yl, 1-piperazinyl, 4-methyl-3-methyl-piperazin-yl, 3-methoxyethyl-piperazin-1-yl, 3-ethyl-piperazin-1-yl, 3-benzyl-piperazin-1-yl or 3-CH₂F-piperazin-1-yl
R₁₅ is Cl, Br, CF₃, F
R₁₆ is CH₃, H, CH₂—CH₃
Rₐ is H or CH₃
R_{b} is H, CH₂—CH₂—CH₃, F, CH(CH₃)₂, Cl, OCH₃, CH₃ or CH₂—CH₃
And when
R is of formula (d)
R₈ is 3-methyl-piperazin-1-yl, 4-benzyl-1-piperazinyl or 1-piperazinyl
R₈ is CH₃ or H
And when
R is of formula (e)
R₉ is -4,7-diaza-spiro[2.5]oct-7-yl, 3-ethyl-piperazin-1-yl, 3-methyl-piperazin-1-yl, 4-methyl-3-methyl-piperazin-1-yl or 3-ethyl-piperazin-1-yl
Rₐ is H, CH₂—CH₃ or CH(CH₃)₂
R_{b} is CH₃, F, CH(CH₃)₂, OCH₃, CH₂—CH₃ or Cl
or a pharmaceutically acceptable salt thereof.

8. A method according to claim 1 wherein the inhibitor is 3-[2-Chloro-5-(4-methyl-piperazin-1-yl)-3-trifluoromethyl-phenyl]-4-(1H-indol-3-yl)-pyrrole-2,5-dione or 3-(1H-Indol-3-yl)-4-[2-(4-methyl-piperazin-1-yl)-quinazolin-4-yl]-pyrrole-2,5-dione;
or a pharmaceutically acceptable salt thereof.

9. A method according to claim 1 wherein a daily dose of 10 to 800 mg of a compound is administered to an adult human.

10. A method according to claim 1 wherein the disorder to be treated is selected from Down's Syndrome, memory and cognitive impairment, dementia, amyloid neuropathies, brain inflammation, nerve and brain trauma, vascular amyloidosis, or cerebral hemorrhage with amyloidosis.

11. A method of treating mammals suffering from neurological and vascular disorders related to beta-amyloid generation and/or aggregation which comprises administering to a said mammal in need of such treatment a pharmaceutical composition comprising
(a) a dose, effective against neurological and vascular disorders related to beta-amyloid generation and/or aggregation, an inhibitor of formula I according to claim 1 or a pharmaceutically acceptable salt thereof and
(b) a therapeutically effective amount of a second drug selected from drugs used to treat neurological and vascular disorders related to beta-amyloid generation and/or aggregation.

12. (canceled)

13. A pharmaceutical composition for use in the treatment of a neurological and vascular disorders related to beta-amyloid generation and/or aggregation comprising an inhibitor of formula I according to claim 1.

14. A method of treating a warm blooded animal having a neurological and vascular disorders related to beta-amyloid generation and/or aggregation comprising administering a therapeutically effective amount of an inhibitor according to claim 1.

15. A combination comprising an inhibitor according to claim 1, and a therapeutically effective amount of a second drug selected from drugs used to treat neurological and vascular disorders related to beta-amyloid generation and/or aggregation.

16. A pharmaceutical composition comprising an inhibitor of formula I 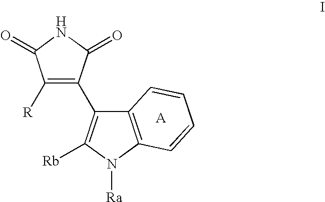
wherein
Rₐ is H; CH₃; CH₂—CH₃; or isopropyl,
R_{b} is H; halogen; C₁₋₆alkoxy; or C₁₋₆alkyl, and either
I. R is a radical of formula (a) 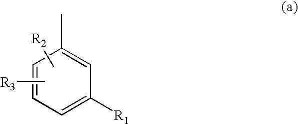
wherein
R1 is piperazin-1-yl optionally substituted by CH₃ in position 3 or 4; or 4,7-diaza-spiro [2.5] oct-7-yl;
R₂ is Cl; Br; CF₃; or CH₃; and
R₃ is H; CH₃; or CF₃; R₃ being other than H when Ra is H or CH₃, Rb is H and R₁ is 4-methyl-1-piperazinyl; or
II. R is a radical of formula (b) 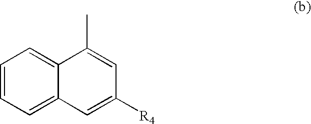
wherein
R₄ is piperazin-1-yl substituted in positions 3 and/or 4 by CH₃; or 4,7-diaza-spiro [2.5] oct-7-yl; Ra being other than H or CH₃ when R₄ is 4-methyl-1-piperazinyl; or
R is a residue of formula (c) 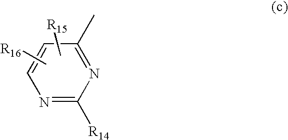
wherein
R₁₄ is piperazin-1-yl optionally substituted by CH₃ in position 3 and/or 4 or in position 3 by ethyl, phenyl-C₁₋₄alkyl, C₁₋₄alkoxy-C1-4alkyl or halogeno-C₁₋₄alkyl; or 4,7-diaza-spiro [2.5] oct-7-yl;
R₁₅ is halogen; CF₃; or CH₃; R₁₅ being other than CH₃ when Ra is H or CH₃, Rb is H and
R₁₄ is 4-methyl-1-piperazinyl; and
R₁₆ is H; CH₃; or CF₃; R₁₆ being other than H when R₁₅ is Cl, Ra is H or CH₃, Rb is H and
R₁₄ is 4-methyl-1-piperazinyl; or
IV. R is a radical of formula (d) 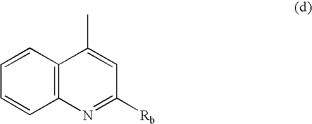
wherein R₈ is piperazin-1-yl, 3-methyl-piperazin-1-yl or 4-benzyl-piperazin-1-yl, or
V. R is a radical of formula (e) 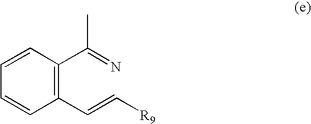
wherein R₉ is 4,7-diaza-spiro [2.5] oct-7-yl; or piperazin-1-yl substituted in position 3 by methyl or ethyl and optionally in position 4 by methyl; or a pharmaceutically acceptable salt thereof in the treatment of neurological and vascular disorders related to beta-amyloid generation and/or aggregation.
